(19) United States
(12) Patent Application Publication
(10) Pub. No.: US 2007/0212398 A1
(43) Pub. Date: Sep. 13, 2007

(54) Powdered wipe

(76) Inventor: Tiffany Aceto, Chester Springs, PA (US)

Correspondence Address: RATNERPRESTIA, P O BOX 980, VALLEY FORGE, PA, 19482-0980, US

(21) Appl. No.: 11/336,120

(22) Filed: Jan. 20, 2006

Related U.S. Application Data
(60) Provisional application No. 60/645,773, filed on Jan. 21, 2005.

(51) Int. Cl. A61K 9/70

(52) U.S. Cl. 424/443

(57) ABSTRACT

A dry wipe to remove sand from a skin surface is provided. The wipe includes a substrate having a hydrophilic powder disposed thereon. The dry wipe is manufactured by the process of providing a wet substrate; applying a hydrophilic powder to at least one side of the wet substrate; and drying the wet substrate. A method of removing sand from a skin surface using the wipe is also provided.

### CROSS-REFERENCE TO RELATED APPLICATION

The present application claims priority from U.S. Provisional Patent Application Ser. No. 60/645,773, filed on 21 Jan. 2005.

### FIELD OF THE INVENTION

The present invention relates to a powder-embedded wipe that is used to wipe sand from a skin surface, a method of manufacturing the wipe, and a method of using the wipe.

### BACKGROUND OF THE INVENTION

Many young children take great joy in playing in sand, whether at the beach or in a sandbox. However, the sand tends to stick to the children's hands, making for a difficult time to clean the child's skin. Additionally, some children tend to touch their faces with sandy hands, transferring some of the sand from their hands 10 onto their faces, making clean-up particularly difficult. Oftentimes, a towel is used to attempt to clean the skin surface, but such application tends only to remove some, but not all of the sand, and the abrasive nature of the sand may cause skin irritation as the sand is rubbed against the skin by the towel.

There exists a need to provide a product that is easy to use to wipe the is sand from a skin surface, such as hands and/or face.

### SUMMARY OF THE INVENTION

Briefly, the present invention provides a dry wipe comprising a substrate having a first side and an opposing second side, with a hydrophilic powder disposed on at least the first side of the substrate.

Also, the present invention provides a dry wipe comprising a substrate having a hydrophilic powder disposed thereon. The dry wipe is manufactured by the process of providing a wet substrate; applying a hydrophilic powder to at least one side of the wet substrate; and drying the wet substrate.

The present application also provides a dry wipe comprising a substrate having a hydrophilic powder disposed thereon. The dry wipe is manufactured by the process of: providing a dry substrate; applying a hydrophilic powder to at least one side of the dry substrate; wetting the dry substrate, thereby forming a wet substrate; and drying the wet substrate.

Further, the present invention provides a method of removing sand from a skin surface comprising providing a dry wipe comprising a substrate having a hydrophilic powder disposed on at least one side of the substrate; and wiping the skin surface and sand on the skin surface with the dry wipe, wherein sand is transferred from the skin surface to the dry wipe.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention. In the drawings:

FIG. 1 is a perspective view of a wipe according to an exemplary embodiment of the present invention.

FIG. 2 is a side profile view of the wipe shown in FIG. 1.

FIG. 3 is a side profile view of an alternative embodiment of a wipe according to the present invention.

FIG. 4 is a top plan view of another alternative embodiment of a wipe according to the present invention.

FIG. 5 is side profile view showing a first method of manufacturing the dry wipe according to the present invention.

FIG. 6 is a side profile view showing a first method of manufacturing the dry wipe according to the present invention.

FIG. 7 is a perspective view showing a third method of manufacturing the dry wipe according to the present invention.

### DETAILED DESCRIPTION OF THE INVENTION

In the drawings, like numerals indicate like elements throughout. In the drawings, like numerals indicate like elements throughout. Certain terminology is used herein for convenience only and is not to be taken as a limitation on the present invention. The terminology includes the words specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

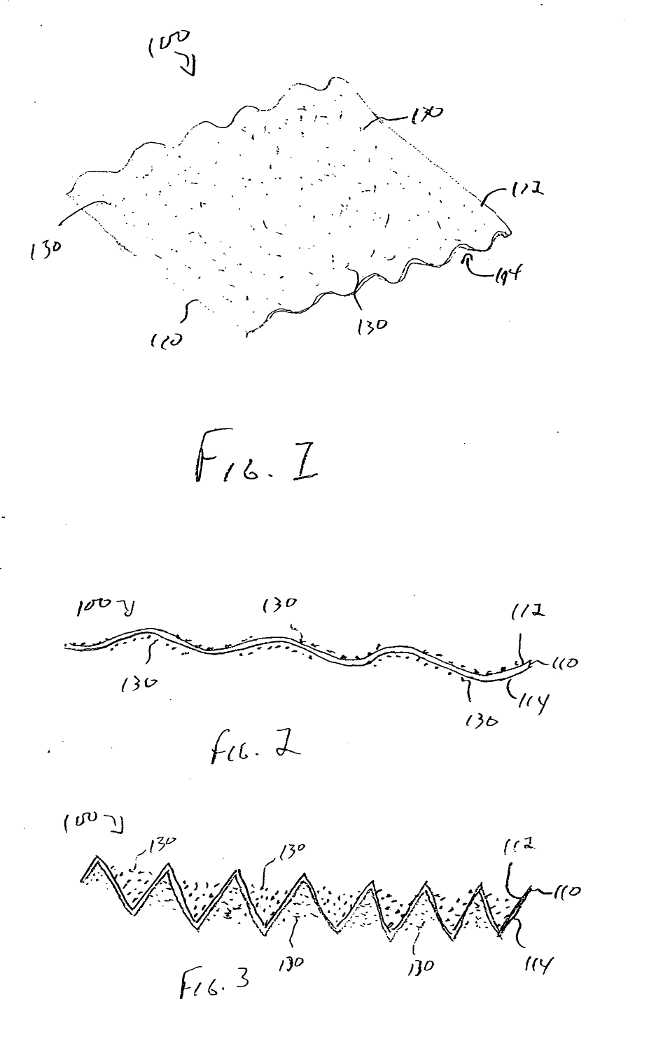

A dry wipe or towelette that is impregnated with a hydrophilic powder, such as talcum powder, is provided. FIG. 1 shows a perspective view of an exemplary embodiment of such a wipe 100. The wipe 100 includes a substrate 110 that is coated or impregnated by the powder 130. The substrate 110 includes a first side 112 and an opposing second side 114. The powder 130 may be disposed on only the first side 112, however the powder 130 may be disposed on both the first side 112 and the second side 114.

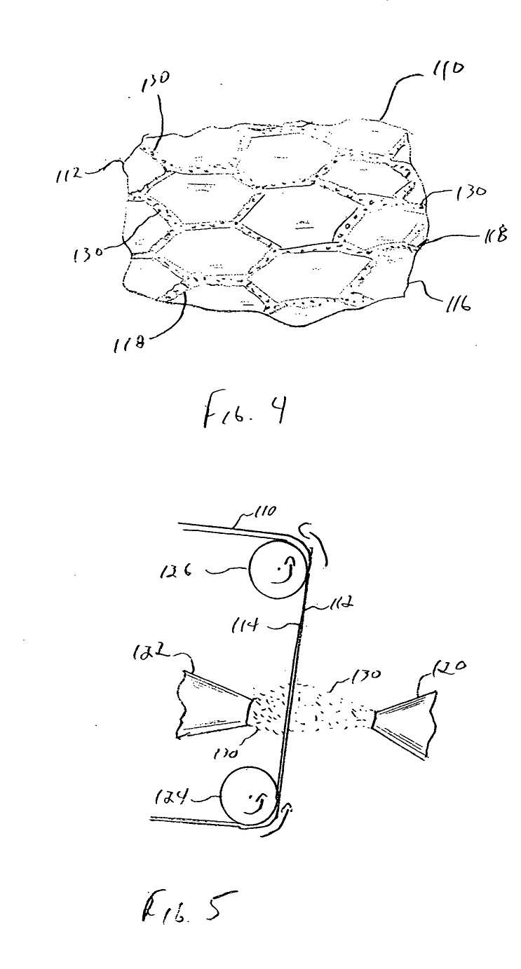

The first and second sides 112, 114 may be contoured to retain particles of the powder 130. As used herein, the term “contoured” means a surface that has non-uniform surface features. Examples of contoured surfaces include embossed, quilted, pleated, corrugated, and ribbed surfaces, although other types of contoured surfaces are contemplated by the present invention. FIGS. 2 and 3 show alternative embodiments of contoured surfaces that may be used with the present invention. FIG. 4 shows a quilted surface 116 with powder 130 embedded in the quilting 118.

The substrate 110 may be woven or non-woven, sponge, foam, battings, balls, puffs, or films. The material comprising the substrate 110 may be natural or synthetic fibers, or a mixture thereof. In an exemplary embodiment, the substrate 110 is constructed from a hydrophilic material such as viscose, cotton, flax, or other suitable hydrophilic material, being natural, synthetic, or a combination thereof.

The substrate 110 may be manufactured according to methods well known to those skilled in the art. For example, the substrate 110 may be manufactured by dry forming techniques such as air-laying or wet laying, such as on a paper making machine. However, the method of manufacturing the substrate 110 is not to be limited by the general methods discussed herein.

The powder 130 may be affixed to the substrate 110 in one of several methods. In one exemplary manufacturing method, the substrate 110 is wetted with a wetting agent, such as water or a volatile solvent, such as alcohol. At least the first side 112, and alternatively, both sides 112, 114, of the substrate 110 are wetted with the wetting agent. The substrate 110 may be in web form, dispensed from a drum or roller, into the wetting agent. After wetting the substrate 110, the powder 130 is applied to at least one side, or alternatively, both sides, of the substrate 110. The wet substrate 110 serves to adhere the powder 130 onto the substrate 110.

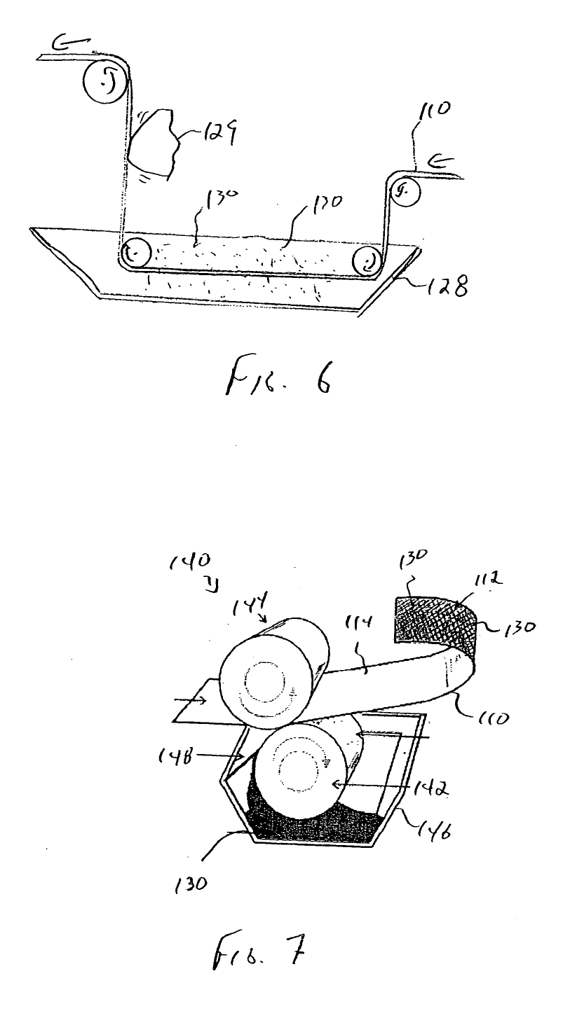

In an alternative exemplary method, illustrated in FIG. 5, the powder 130 is dry sprayed onto the wet substrate 110. The powder 130 may be sprayed only onto the first surface 112, or alternatively, the powder 130 is sprayed onto both the first and second surfaces 112, 114, as shown in FIG. 5. Spray nozzles 120, 122 are disposed on either side of the substrate 110, which is drawn between rollers 124, 126. As the substrate 110 is pulled between the rollers 124, 126, the nozzles 120, 122 spray the powder 130 onto each of the first and second sides 112, 114 of the substrate 110, respectively.

In another exemplary method of manufacturing the dry wipe 100, shown in FIG. 6, the substrate 110 is wetted and then drawn through a pan 128 containing the powder 130. Excess powder 130 is shaken from the substrate 110, such as by vibrating the substrate 110 with a vibrator 129.

In another method of manufacturing the dry wipe 100, shown in FIG. 7, the substrate 110 is wetted as described above and then run through a gravure drum assembly 140. The substrate 110 is run over a rotating gravure drum 142 loaded with powder 130 to force powder 130 into the substrate 110. The substrate 110 is forced between the rotating gravure drum 142 and a rotating impression cylinder 144. As the gravure drum 142 rotates, the gravure drum 142 picks up powder 130 disposed in a vessel 146. A doctor blade 148 removes excess powder 130 from the gravure drum 142 prior to contact with the substrate 110. As the substrate 110 engages the powder-loaded drum 142, the impression cylinder 144 forces the powder 130 onto the substrate 110 SO that the powder 130 is embedded in the first side 112 of the substrate 110, forming the wipe 100. If the powder 130 is to be embedded on the second side 114 of the substrate 110, the process is repeated on a second gravure drum assembly (not shown). Although not shown, the gravure drum 142 may be contoured to impose a contoured surface to the substrate 110 as the substrate 110 is drawn over the gravure drum 142.

In another exemplary method of applying the powder 130 to the substrate 110, the powder 130 is wetted with a wetting agent, such as water or a volatile liquid, such as alcohol. The wet powder 130 is then sprayed onto the dry substrate 110, forming the wipe 100. The arrangement for spraying the wetted powder 130 onto the substrate 110 is similar to that shown in FIG. 5, but the powder 130 being sprayed from the nozzles 120, 122 is pre-wetted prior to the powder 130 engaging the substrate 110.

In yet another exemplary method of applying powder 130 to the substrate 110, dry powder 130 is applied to a dry substrate 110 in at least one of any of the methods described above. The dry substrate 110 and dry powder 130 are then wetted down to adhere the powder 130 onto the substrate 110. After the powder 130 is applied to the substrate 110, the substrate 110 and the powder 130 on the substrate 110 are dried, such as by heating or other suitable method. The powder 130 remains affixed to the now-dried substrate 110.

The substrate 110 can then be cut into appropriately sized sheets, forming a plurality of wipes 100. The wipes 100 are stored in a container (not shown) for dispensing, such as in a Z-fold configuration as disclosed by U.S. Pat. No. 6,213,344, in a spiral configuration as disclosed by U.S. Pat. No. 4,817,790, or any other suitable configuration conducive to quick and easy dispensing.

The methods recited above for adhering the powder 130 to the substrate 110 are illustrative only and are not meant to be limiting in any way. Those skilled in the art will recognize that alternative methods of applying the powder 130 to the substrate 110 may be used, without departing from the spirit and scope of the present invention. However, it is within the scope and spirit of the present invention that the finished product be a dry wipe 100 having the substrate 110 with the powder 130 adhered thereto or embedded therein.

To use the wipe 100, the wipe 100 is removed from its storage container and rubbed along a sandy skin surface, such as hands or face. The water in the sand and on the skin surface that serves to adhere the sand to the skin surface is dried by the powder 130. The sand is thus transferred from the skin surface to the substrate 110, and the substrate 110 wipes the sand off of the skin surface. After use, the wipe 100 is discarded.

While an exemplary use for the wipe 100 is to clean sand from a skin surface, it has been recognized that the absorbent capabilities of the powder 130 impregnated within the substrate 110 may also be utilized to absorb excess moisture from the skin surface, particularly in areas of skin-to-skin contact, such as under arms, in the crotch area, between toes, or any other skin surface areas that may need to be dried.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed:

1. A dry wipe comprising:
a substrate having a first side and an opposing second side; and
a hydrophilic powder disposed on at least the first side of the substrate.

2. The dry wipe according to claim 1, wherein the hydrophilic powder is further disposed on the second side of the substrate.

3. The dry wipe according to claim 1, wherein the hydrophilic powder comprises talcum powder.

4. The dry wipe according to claim 1, wherein the substrate comprises a contoured surface.

5. A dry wipe comprising a substrate having a hydrophilic powder disposed thereon, wherein the dry wipe is manufactured by the process of:
providing a wet substrate;
applying a hydrophilic powder to at least one side of the wet substrate; and
drying the wet substrate.

6. The dry wipe according to claim 5, wherein applying the hydrophilic powder to at least one side of the wet substrate comprises applying the hydrophilic powder to two sides of the wet substrate.

7. The dry wipe according to claim 5, wherein the substrate comprises a contoured surface.

8. The dry wipe according to claim 5, wherein applying the hydrophilic powder comprises spraying the hydrophilic powder onto the substrate.

9. The dry wipe according to claim 5, wherein applying the hydrophilic powder comprises advancing the wet substrate through a volume of powder and removing excess powder from the substrate.

10. The dry wipe according to claim 5, wherein the hydrophilic powder comprises talcum powder.

11. A dry wipe comprising a substrate having a hydrophilic powder disposed thereon, wherein the dry wipe is manufactured by the process of:
providing a wet substrate;
applying a hydrophilic powder to at least one side of the dry substrate;
wetting the dry substrate, thereby forming a wet substrate; and
drying the wet substrate.

12. The dry wipe according to claim 11, wherein applying the hydrophilic powder to at least one side of the dry substrate comprises applying the hydrophilic powder to two sides of the dry substrate.

13. The dry wipe according to claim 11, wherein the substrate comprises a contoured surface.

14. The dry wipe according to claim 11, wherein the hydrophilic powder comprises talcum powder.

15. A dry wipe comprising a substrate having a hydrophilic powder disposed thereon, wherein the dry wipe is manufactured by the process of:
providing a dry substrate;
wetting a hydrophilic powder;
applying the hydrophilic powder to at least one side of the dry substrate; and
drying the wet hydrophilic powder on the substrate.

16. The dry wipe according to claim 15, wherein applying the hydrophilic powder to at least one side of the dry substrate comprises applying the hydrophilic powder to two sides of the dry substrate.

17. The dry wipe according to claim 15, wherein the substrate comprises a contoured surface.

18. The dry wipe according to claim 15, wherein the hydrophilic powder comprises talcum powder.

19. A method of removing sand from a skin surface comprising the steps of:
providing a dry substrate;
providing a dry wipe comprising a substrate having a hydrophilic powder disposed on at least one side of the substrate; and
wiping the skin surface and sand on the skin surface with the dry wipe, wherein sand is transferred from the skin surface to the dry wipe.
